(19) United States
(12) Patent Application Publication
(10) Pub. No.: US 2021/0315937 A1
(43) Pub. Date: Oct. 14, 2021

(54) METHODS OF TREATING INFLAMMATION

(71) Applicant: University of South Florida, Tampa, FL (US)

(72) Inventors: Subhra Mohapatra, Lutz, FL (US); Shyam S. Mohapatra, Lutz, FL (US); Mahasweta Das, Tampa, FL (US); Andrew McGill, Tampa, FL (US); Taylor Martinez, Tampa, FL (US); Karthick Mayilsamy, Tampa, FL (US)

(21) Appl. No.: 17/224,831

(22) Filed: Apr. 7, 2021

Related U.S. Application Data
(60) Provisional application No. 63/006,835, filed on Apr. 8, 2020.
(60) Provisional application No. 63/007,236, filed on Apr. 8, 2020.

(51) Int. Cl. A61K 35/28; A61K 31/4439; A61K 9/00

(52) U.S. Cl. CPC A61K 35/28; A61K 9/0019; A61K 9/0043; A61K 31/4439

(57) ABSTRACT

The present application is related to methods of treating ARDS in a subject in need thereof, by administering (a) pioglitazone, or a pharmaceutically acceptable salt thereof, and (b) mesenchymal stromal cells to the subject. Also disclosed herein are methods of treating one or more symptoms of ARDS in a subject in need thereof.

### CROSS-REFERENCE TO RELATED APPLICATION

This application claims priority to U.S. Provisional Patent Application No. 63/006,835, and U.S. Provisional Patent Application No. 63/007,236, both filed Apr. 8, 2020, which are incorporated herein by reference in their entirety.

### TECHNICAL FIELD

This application relates to methods of treating inflammation in multi-organ human diseases, particularly using anti-inflammatories such as pioglitazone (PG) by itself or in combination with mesenchymal stromal cells (MSC).

### BACKGROUND

The current global pandemic of coronavirus disease 2019 (COVID-19) is caused by the Severe Acute Respiratory Syndrome Coronavirus-2 (SARS-CoV-2), which has afflicted >1.1 million people across 205 countries with >60,000 deaths. It is projected to increase to ˜1.4 million by April 30 with 100,000 deaths. SARS-CoV-2 is an enveloped non-segmented positive-strand RNA virus, which shares 80% homologies with SARS-CoV. The SARS-CoV-2 infection causes pneumonia leading to acute respiratory distress syndrome (ARDS) and similar to SARS-CoV or Middle Eastern Respiratory Syndrome CoV (MERS-CoV). See Song Z, et al., Viruses. 2019; 11(1). In addition to respiratory symptoms, SARS-CoV-2 infected patients show neurological manifestations (see Mao, et al., medRxiv. 2020; 2020.02.22.20026500); 78 (36.4%) out of 214 patients including 6% showing symptoms of stroke and 15% showing encephalopathy or syncope. See Tape, et al., RI Med J. 2020; 103(3):50-1. At present, there is no vaccine or therapy for COVID-19, though a number of investigational trials are underway, including repurposing of remdesivir, and infusion of immunoglobulin from recovered COVID-19 patients. [See Jawhara, Int J Mol Sci. 2020; 21(7)]. However, none of these potential therapies address the neuronal damage caused by the SARS-CoV-2 infection. Hence, there is an urgent dire need to find a suitable treatment strategy, which will reduce the virus-induced neuronal damages and mortality.

Several human β-coronaviruses including SARS-CoV, MERS-CoV, HCoV-229E, and HCoV-OC43 have shown neuroinvasive properties. Studies on samples isolated from SARS patients in 2002-03 showed the presence of the virus in the brain, located exclusively in the neurons. See Xu, et al., Clin Infect Dis. 2005; 41(8):1089-96; and Li, et al., J Med Virol. 2020. For example, SARS-CoV-2 was observed in the cerebrospinal fluid (CSF) of a SARS patient with acute respiratory distress syndrome (ARDS) (see Baig, et al., ACS Chem Neurosci. 2020) and certain COVID-19 patients showed neuropathy along with hyposmia and altered taste sensation. In addition, the presence of SARS-CoV has also been demonstrated in mouse brain with low-grade infection experimentally infected with SARS (see Netland, et al., J Virol. 2008; 82(15):7264-75), suggesting that coronaviruses transported to the brain from the peripheral organs like lungs or the nasal epithelium increases the likelihood of CNS infections. Together, these reports suggest that in addition to respiratory illness, SARS-CoV-2 significantly affects the CNS, particularly brain stem respiratory centers (BSRC). Indeed, other β-coronaviruses, SARS-CoV-2 can infect the brain stem either through the olfactory tract (nose-to-brain axis) and cause brain damage with the potential to cause neuro-physiological and -behavioral complications leading to severe functional deficits and even death. Also, it has been suggested that SARS-CoV-2 infection may cause neuronal death affecting brain medulla and pons, which are regulators of voluntary and involuntary breathing, leading to system failure and patient's loss of ability to breathe.

Current therapies against critically ill patients suffering from ARDS include treatment for lung inflammation. However, as noted above, ARDS may result from a viral infection impacting the brain stem respiratory center (BSRC), which may not be ameliorated by standard viral or anti-inflammatory therapies.

### SUMMARY

The present application is based, in part, on the surprising and unexpected discovery that reducing neuroinflammation and neurodegeneration in the brain, may contribute to treating certain respiratory disorders such as ARDS. Some embodiments described herein provide methods of treating a patient experiencing symptoms associated with ARDS.

Some embodiments described herein provide methods of treating one or more symptoms of ARDS in a subject in need thereof, comprising administering (a) pioglitazone, or a pharmaceutically acceptable salt thereof, and (b) mesenchymal stromal cells.

Some embodiments described herein provide methods of treating ARDS in a subject in need thereof, comprising administering (a) pioglitazone, or a pharmaceutically acceptable salt thereof, and (b) mesenchymal stromal cells.

Some embodiments described herein provide methods of reducing neuroinflammation in a subject in need thereof, comprising administering (a) pioglitazone, or a pharmaceutically acceptable salt thereof, and (b) mesenchymal stromal cells; wherein the subject has been previously diagnosed with a viral infection.

Some embodiments described herein provide methods of treating ARDS in a subject in need thereof, comprising administering (a) pioglitazone, or a pharmaceutically acceptable salt thereof, and (b) mesenchymal stromal cells.

The details of one or more embodiments of the disclosure are set forth in the accompanying drawings and the description below. Other features, objects, and advantages of the disclosure will be apparent from the description and drawings, and from the claims.

### BRIEF DESCRIPTION OF FIGURES

FIG. 1 illustrates the anti-inflammatory effects of pioglitazone in a HEK293 cell model.

FIG. 2 shows relative fold change in RNA expression of various inflammatory genes in LPS (100 ng/ml) challenged HEK293 cells. The cells were stimulated with LPS for 24 hrs and then treated with PG (20 mM) for 24 hrs. *p<0.05, t test.

FIG. 3 shows that PG and MSC (conditioned media) combination treatment reduces LPS-induced inflammation in IMG cells. 3A) Immunofluorescence images showing the Ibal expression (red); DAPI (blue). 3B) ImageJ quantitation of Ibal immunoreactivity. **p<0.001.

FIG. 4 shows fold changes in mRNA expressions of in pro-inflammatory (Il-b, IL-6, TNFa, CCL20) and anti-inflammatory (PPARg and IL-10) by qPCR*p<0.05, **p<0.01, ***p<0.001. Vehicle (V), LPS (L), LPS+PG (L+P), LPS+MSC (L+M) or LPS+PG+MSC (L+P+M).

### DETAILED DESCRIPTION

Unless otherwise defined, all technical and scientific terms used herein have the same meaning as commonly understood by one of ordinary skill in the art to which this disclosure belongs. Methods and materials are described herein for use in the present application; other, suitable methods and materials known in the art in some aspects this disclosure are also used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control. When trade names are used herein, the trade name includes the product formulation, the generic drug, and the active pharmaceutical ingredient(s) of the trade name product, unless otherwise indicated by context.

As used herein, terms “treat” or “treatment” refer to preventive, therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the term “subject,” refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of ARDS. In some embodiments, the subject has been identified or diagnosed as having ARDS (e.g., as determined using a regulatory agency-approved, e.g., FDA-approved, assay or kit, or the standard of care diagnostic). In some embodiments, the subject is suspected of having ARDS.

The term “regulatory agency” refers to a country's agency for the approval of the medical use of pharmaceutical agents with the country. For example, a non-limiting example of a regulatory agency is the U.S. Food and Drug Administration (FDA).

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

Combination of pioglitazone, a PPAR-γ agonist that has been FDA-approved for the treatment of diabetes, and intranasally administered mesenchymal stem cells (MSCs) provide effective therapy in traumatic brain injury (TBI) by respectively contributing to their anti-inflammatory and neuro-regenerative activities. These results suggest that PG provides a suitable anti-inflammatory micro-environment for MSC to exert their optimum neuro-regenerative functions. See Das, et al., Rev Neurosci. 2019; 30(8):839-55 and Sci Rep. 2019; 9(1):13646. Further, others have reported that PG also possesses profound antiviral properties and it provides protection against RNA viruses, such as HIV, rotavirus. See Chojkier, et al, PLoS One. 2012; 7(3):e31516; Guerrero, et al., Antiviral Res. 2012; 96(1):1-12; Khattab, et al, Liver Int. 2010; 30(3):447-54; and Omeragic, et al., Sci Rep. 2019; 9(1):9428.

The term “salt,” as used herein, refers to organic or inorganic salts of a compound, such as pioglitazone, as described herein. Exemplary salts include, but are not limited to, sodium, potassium, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A salt may involve the inclusion of another molecule such as an acetate ion, a succinate ion or other counterion. The counterion may be any organic or inorganic moiety that stabilizes the charge on the parent compound. Furthermore, a salt has one or more than one charged atom in its structure. In instances where there are multiple charged atoms as part of the salt multiple, counter ions are sometimes present. Hence, a salt can have one or more charged atoms and/or one or more counterions. A “pharmaceutically acceptable salt” is one that is suitable for administration to a subject as described herein and in some aspects includes salts as described by P. H. Stahl and C. G. Wermuth, editors, Handbook of Pharmaceutical Salts:

Properties, Selection and Use, Weinheim/Zürich:Wiley-VCH/VHCA, 2002, the list for which is specifically incorporated by reference herein.

In relation to COVID-19, transplantation of ACE2-mesenchymal stem cells was shown to improve the outcome of patients with COVID-19 pneumonia, in which all 7/7 critically ill patients showed improvement of pulmonary functions and symptoms without any observed side effects 2 days post-transplantation. See Leng, et al. Aging and disease. 2020((2): 216-228). Since mortality in COVID-19 patients with the inflammatory lung condition and ARDS is ˜50% and is associated with older age, co-morbidities such as diabetes, higher disease severity, and elevated markers of inflammation. See Liu, et al., medRxiv. 2020. doi: 10.1101/2020.02.17.20024166.

The present disclosure is based in part on the surprising and unexpected discovery that treatment with a combination of pioglitazone, or a pharmaceutically acceptable salt thereof, and MSC therapy, as described herein may reduce disease symptoms and improve patient outcomes in subjects having viral infections such as COVID-19. Without being bound by any theory, the SARS-CoV-2 virus may migrate from the nasal epithelium to the BSRC, resulting in respiratory distress (such as ARDS) or respiratory failure. The combination of pioglitazone, or a pharmaceutically acceptable salt thereof, and MSC therapy, as described herein may reduce the viral load in the subject while attenuating the viral-induced neuroinflammation.

MSCs are pluripotent cells that are present in a wide variety of body tissues (see Da Silva et al., J Cell Sci 2006, 119, 2204-2213 and Karp and Leng Teo, Cell Stem Cell 2009, 4, 206-216) and can be isolated and cultured, and may ultimately differentiate into many kinds of cells (see Hasan, et al. Front Neurol 2017, 8, 28; Pittenger, et al. Science 1999, 284, 143-147, and Sanchez-Ramos, et al. Exp Neurol 2000, 164, 247-256). Their immune tolerance (see Galindo, et al. Neurol Res Int 2011, 564089), ability to migrate to the site of inflammation (see Alexanian, et al., Neurorehabil Neural Repair 2011, 25, 873-880; Chamberlain, et al., Stem Cells 2007, 25, 2739-2749; and Chamberlain, et al., PLoS One 2011, 6, e25663), and secretion of growth promoting factors (see Parr, et al., Bone Marrow Transplant. 2007, 40, 609-619 and Redondo-Castro, et al., Stem Cell Res Ther 2017, 8, 79) makes them a candidate for regenerative therapies.

Some embodiments provide a method of treating ARDS in a subject in need thereof, comprising administering (a) pioglitazone, or a pharmaceutically acceptable salt thereof, and (b) mesenchymal stromal cells.

Other embodiments provide a method of treating one or more symptoms of ARDS in a subject in need thereof, comprising administering (a) pioglitazone, or a pharmaceutically acceptable salt thereof, and (b) mesenchymal stromal cells.

In some embodiments, the subject has been previously diagnosed with a viral infection. In some embodiments, the viral infection is a coronavirus infection. In some embodiments, the viral infection is SARS-COV2 (COVID-19).

Some embodiments described herein provide methods of reducing neuroinflammation in a subject in need thereof, comprising administering (a) pioglitazone, or a pharmaceutically acceptable salt thereof, and (b) mesenchymal stromal cells; wherein the subject has been previously diagnosed with a viral infection. In some embodiments, the viral infection is a coronavirus infection. In some embodiments, the viral infection is SARS-COV2 (COVID-19).

In some embodiments, (a) is administered prior to (b). In other embodiments, (a) is administered prior to, and concurrently with, (b).

In some embodiments, wherein the administration of (a) pioglitazone, or a pharmaceutically acceptable salt thereof, is between about 1 day and about 10 days prior to the administration of (b) mesenchymal stromal cells. For example, in some embodiments, the pioglitazone, or a pharmaceutically acceptable salt thereof, is administered about 1 day, about 2 days, about 3 days, about 4 days, about 5 days, about 6 days, about 7 days, about 8 days, about 9 days, or about 10 days, prior to the administration of (b) mesenchymal stromal cells, or any value in between. In some embodiments, wherein the administration of (a) pioglitazone, or a pharmaceutically acceptable salt thereof, is between about 3 days and about 7 days prior to the administration of (b) mesenchymal stromal cells.

In some embodiments, wherein the administration of (a) pioglitazone, or a pharmaceutically acceptable salt thereof, is between about 30 minutes and about 24 hours prior to the administration of (b) mesenchymal stromal cells. For example, in some embodiments, the pioglitazone, or a pharmaceutically acceptable salt thereof, is administered about 30 minutes, about 45 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 7 hours, about 8 hours, about 9 hours, about 10 hours, about 11 hours, about 12 hours, about 14 hours, about 16 hours, about 18 hours, about 20 hours, about 22 hours, or about 24 hours, prior to the administration of (b) mesenchymal stromal cells, or any value in between. In other embodiments, the administration of (a) pioglitazone, or a pharmaceutically acceptable salt thereof, is between about 30 minutes and about 12 hours prior to the administration of (b) mesenchymal stromal cells. For example, in some embodiments, the pioglitazone, or a pharmaceutically acceptable salt thereof, is administered about 30 minutes, about 45 minutes, about 60 minutes, about 90 minutes, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, prior to the administration of (b) mesenchymal stromal cells, or any value in between. In still other embodiments, the administration of (a) pioglitazone, or a pharmaceutically acceptable salt thereof, is between about 30 minutes and about 2 hours prior to the administration of (b) mesenchymal stromal cells. For example, about 30 minutes, about 40 minutes, about 50 minutes, about 60 minutes, about 70 minutes, about 80 minutes, about 90 minutes, about 100 minutes, about 110 minutes, about 2 hours, prior to the administration of (b) mesenchymal stromal cells, or any value in between.

In some embodiments, between about 50 mg and about 1,000 mg of pioglitazone, or a pharmaceutically acceptable salt thereof is administered to the subject. For example, about 50 mg, about 100 mg, about 200 mg, about 300 mg, about 400 mg, about 500 mg, about 600 mg, about 700 mg, about 800 mg, about 900 mg, or about 1,000 mg, of pioglitzone, or a pharmaceutically acceptable salt thereof is administered to the subject, or any value in between. In other embodiments, between about 100 mg and 750 mg of pioglitazone, or a pharmaceutically acceptable salt thereof is administered to the subject. For example, about 100 mg, about 200 mg, about 300 mg, about 400 mg, about 500 mg, about 600 mg, about 700 mg, about 750 mg, of pioglitzone, or a pharmaceutically acceptable salt thereof is administered to the subject, or any value in between. In still other embodiments, between about 250 mg and 500 mg of pioglitazone, or a pharmaceutically acceptable salt thereof is administered to the subject. For example, about 250 mg, about 300 mg, about 350 mg, about 400 mg, about 450 mg, about 500 mg, of pioglitzone, or a pharmaceutically acceptable salt thereof is administered to the subject, or any value in between.

In some embodiments, between about 1 mg and about 10 mg of pioglitazone, or a pharmaceutically acceptable salt thereof, per kilogram of body weight, is administered to the subject, or any value in between. For example, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg, of pioglitzone, or a pharmaceutically acceptable salt thereof is administered to the subject, or any value in between.

In some embodiments, the pioglitazone, or a pharmaceutically acceptable salt thereof is administered orally. In some embodiments, the pioglitazone, or a pharmaceutically acceptable salt thereof is administered intravenously. In some embodiments, the pioglitazone, or a pharmaceutically acceptable salt thereof is administered intranasally.

In some embodiments, the mesenchymal stromal cells are administered intranasally. In other embodiments, the mesenchymal stromal cells are administered intravenously. In still other embodiments, the mesenchymal stromal cells are administered intra-arterially. In further embodiments, the mesenchymal stromal cells are administered intra-cranially.

Some embodiments further comprise administering (a) pioglitazone, or a pharmaceutically acceptable salt thereof, after the administration of (b) mesenchymal stromal cells. In some embodiments, the pioglitazone, or a pharmaceutically acceptable salt thereof, is administered from about 1 hour to about 4 weeks after the mesenchymal stromal cells, for example, about 1 hour, about 2 hours, about 3 hours, about 4 hours, about 5 hours, about 6 hours, about 7 hours, about 8, hours, about 9 hours, about 10 hours, about 11 hours, about 12 hours, about 14 hours, about 16 hours, about 18 hours, about 20 hours, about 22 hours, about 24 hours, about 36 hours, about 48 hours, about 60 hours, about 72 hours, about 96 hours, about 5 days, about 6 days, about 7 days, about 8 days, about 9 days, about 10 days, about 2 weeks, about 3 weeks, about 4 weeks, or any value in between. In some embodiments, the pioglitazone, or a pharmaceutically acceptable salt thereof, is administered for between about 1 day to about 7 days after the mesenchymal stromal cells.

In some embodiments, about 2×10⁷ to about 1.5×10⁹ mesenchymal stromal cells are administered to the subject. For example, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, 7×10⁸, about 8×10⁸, about 9×10⁸, about 1×10⁹, or about 1.5×10⁹ mesenchymal stromal cells are administered to the subject, or any value in between.

In some embodiments, about 0.5×10⁶ to about 1×10⁷ mesenchymal stromal cells are administered to the subject per kilogram of body weight. For example, about 0.5×10⁶, about 0.6×10⁶, about 0.7×10⁶, about 0.8×10⁶, about 0.9×10⁶, about 1×10⁶, about 1.1×10⁶, about 1.2×10⁶, about 1.3×10⁶, about 1.4×10⁶, about 1.5×10⁶, about 1.6×10⁶, about 1.7×10⁶, about 1.8×10⁶, about 1.9×10⁶, about 1×10⁷, mesenchymal stromal cells are administered to the subject per kilogram of body weight, or any value in between.

In some embodiments, the pioglitazone, or a pharmaceutically acceptable salt thereof, and the mesenchymal stromal cells are both administered intranasally in separate dosage forms. In some embodiments, the pioglitazone, or a pharmaceutically acceptable salt thereof, and the mesenchymal stromal cells are both administered intranasally in a fixed-dose combination.

Some embodiments provide a method of treating ARDS in a subject in need thereof, comprising administering (a) pioglitazone, or a pharmaceutically acceptable salt thereof, and (b) mesenchymal stromal cells; wherein the (a) pioglitazone, or a pharmaceutically acceptable salt thereof, is administered orally prior to intranasal administration of the (b) mesenchymal stromal cells. Other embodiments provide a method of treating one or more symptoms of ARDS in a subject in need thereof, comprising administering (a) pioglitazone, or a pharmaceutically acceptable salt thereof, and (b) mesenchymal stromal cells; wherein the (a) pioglitazone, or a pharmaceutically acceptable salt thereof, is administered orally prior to intranasal administration of the (b) mesenchymal stromal cells.

Some embodiments provide a method of treating neuroinflammation in a subject in need thereof, comprising administering (a) pioglitazone, or a pharmaceutically acceptable salt thereof, and (b) mesenchymal stromal cells; wherein the (a) pioglitazone, or a pharmaceutically acceptable salt thereof, is administered orally prior to intranasal administration of the (b) mesenchymal stromal cells; wherein the subject has been previously diagnosed with a viral infection. Other embodiments provide a method of treating one or more symptoms of ARDS in a subject in need thereof, comprising administering (a) pioglitazone, or a pharmaceutically acceptable salt thereof, and (b) mesenchymal stromal cells; wherein the (a) pioglitazone, or a pharmaceutically acceptable salt thereof, is administered orally prior to intranasal administration of the (b) mesenchymal stromal cells wherein the subject has been previously diagnosed with a viral infection. In some embodiments, the viral infection is a coronavirus infection. In some embodiments, the viral infection is SARS-COV2 (COVID-19).

In some embodiments, the one or more symptoms are selected from: cough, shortness of breath, fast breathing, and low blood oxygen levels.

In some embodiments, the one or more symptoms comprise two or more symptoms. In other embodiments, the one or more symptoms comprise three or more symptoms. In still other embodiments, the one or more symptoms comprise two to four symptoms.

Some embodiments provide a method of treating a patient experiencing symptoms associated with ARDS as substantially disclosed herein. Other embodiments provide a method of improving the efficacy of stem cells by administering an anti-inflammatory drug as substantially disclosed herein. In some embodiments, the anti-inflammatory drug is pioglitazone.

In some embodiments, the subject has been previously determined to have ARDS. In some embodiments, the subject has been previously diagnosed as having a ARDS. In some embodiments, the subject is currently suffering from ARDS. In some embodiments, the subject is suspected to have ARDS.

In some embodiments, the expression of inflammatory cytokines in a subject is reduced after administering pioglitazone, or a pharmaceutically acceptable salt thereof, and mesenchymal stromal cells to the subject.

In some embodiments, the expression of one or more RNA transcripts encoding one or more inflammatory cytokines in a subject is decreased after administering pioglitazone, or a pharmaceutically acceptable salt thereof, and mesenchymal stromal cells to the subject. In some embodiments, the expression of one or more RNA transcripts encoding one or more inflammatory cytokines is decreased by about 10% to about 90% after administering pioglitazone, or a pharmaceutically acceptable salt thereof, and mesenchymal stromal cells, for example, about 10%, about 15%, about 20%, about 25%, about 30%, about 35%, about 40%, about 45%, about 50%, about 55%, about 60%, about 65%, about 70%, about 75%, about 80%, about 85%, or about 90%.

In some embodiments, the activity of one or more inflammatory cytokines in a subject is decreased after administering pioglitazone, or a pharmaceutically acceptable salt thereof, and mesenchymal stromal cells to the subject. In some embodiments, the activity of one or more inflammatory cytokines is decreased by about 10% to about 90% after administering pioglitazone, or a pharmaceutically acceptable salt thereof, and mesenchymal stromal cells, for example, about 10%, about 15%, about 20%, about 25%, about 30%, about 35%, about 40%, about 45%, about 50%, about 55%, about 60%, about 65%, about 70%, about 75%, about 80%, about 85%, or about 90%.

In some embodiments, the one or more inflammatory cytokines are independently selected from the group consisting of: CCL20, IFNβ, TNFα, IL-6, IL-1ra, IL-4, IL-10, IL-11, IL-13, and TGFβ. In some embodiments, the inflammatory cytokine is CCL20. In some embodiments, the inflammatory cytokine is IFNβ. In some embodiments, the inflammatory cytokine is TNFα. In some embodiments, the inflammatory cytokine is IL-6. In some embodiments the expression and/or activity of CCL20, TNFα, and/or IL-6 are decreased. In some embodiments, the expression and/or activity of CCL20 is decreased. In some embodiments, the expression and/or activity of TNFα is decreased. In some embodiments, the expression and/or activity of IL-6 is decreased.

In some embodiments, the expression of one or more inflammasome associated proteins in a subject is decreased after administering pioglitazone, or a pharmaceutically acceptable salt thereof, and mesenchymal stromal cells to the subject. In some embodiments, the expression of one or more inflammasome associated proteins is decreased by about 10% to about 90% after administering pioglitazone, or a pharmaceutically acceptable salt thereof, and mesenchymal stromal cells, for example, about 10%, about 15%, about 20%, about 25%, about 30%, about 35%, about 40%, about 45%, about 50%, about 55%, about 60%, about 65%, about 70%, about 75%, about 80%, about 85%, or about 90%.

In some embodiments, the activity of one or more inflammasome associated proteins in a subject is decreased after administering pioglitazone, or a pharmaceutically acceptable salt thereof, and mesenchymal stromal cells to the subject. In some embodiments, the activity of one or more inflammasome associated proteins is decreased by about 10% to about 90% after administering pioglitazone, or a pharmaceutically acceptable salt thereof, and mesenchymal stromal cells, for example, about 10%, about 15%, about 20%, about 25%, about 30%, about 35%, about 40%, about 45%, about 50%, about 55%, about 60%, about 65%, about 70%, about 75%, about 80%, about 85%, or about 90%.

In some embodiments, the expression of one or more inflammasome associated proteins in a subject is increased after administering pioglitazone, or a pharmaceutically acceptable salt thereof, and mesenchymal stromal cells to the subject. In some embodiments, the expression of one or more inflammasome associated proteins is increased by about 10% to about 200% after administering pioglitazone, or a pharmaceutically acceptable salt thereof, and mesenchymal stromal cells, for example, about 10%, about 15%, about 20%, about 25%, about 30%, about 35%, about 40%, about 45%, about 50%, about 55%, about 60%, about 65%, about 70%, about 75%, about 80%, about 85%, about 90%, about 100%, about 105%, about 110%, about 115%, about 120%, about 125%, about 130%, about 125%, about 130%, about 145%, about 150%, about 155%, about 160%, about 165%, about 170%, about 175%, about 180%, about 195%, about 190%, or about 200%.

In some embodiments, the activity of one or more inflammasome associated proteins in a subject is increased after administering pioglitazone, or a pharmaceutically acceptable salt thereof, and mesenchymal stromal cells to the subject. In some embodiments, the activity of one or more inflammasome associated proteins is increased by about 10% to about 200% after administering pioglitazone, or a pharmaceutically acceptable salt thereof, and mesenchymal stromal cells, for example, about 10%, about 15%, about 20%, about 25%, about 30%, about 35%, about 40%, about 45%, about 50%, about 55%, about 60%, about 65%, about 70%, about 75%, about 80%, about 85%, about 90%, about 100%, about 105%, about 110%, about 115%, about 120%, about 125%, about 130%, about 125%, about 130%, about 145%, about 150%, about 155%, about 160%, about 165%, about 170%, about 175%, about 180%, about 195%, about 190%, or about 200%.

In some embodiments, PPARγ and/or IL-10 gene expressions increases. In some embodiments, PPARγ and/or IL-10 gene expressions increases after PG or PG-hMSC treatments. In some embodiments, the gene expression increases by about 10% to about 200% after administering pioglitazone, or a pharmaceutically acceptable salt thereof, and mesenchymal stromal cells, for example, about 10%, about 15%, about 20%, about 25%, about 30%, about 35%, about 40%, about 45%, about 50%, about 55%, about 60%, about 65%, about 70%, about 75%, about 80%, about 85%, about 90%, about 100%, about 105%, about 110%, about 115%, about 120%, about 125%, about 130%, about 125%, about 130%, about 145%, about 150%, about 155%, about 160%, about 165%, about 170%, about 175%, about 180%, about 195%, about 190%, or about 200%.

In some embodiments, one or more inflammasome associated proteins are selected from the group consisting of: IL-1β, NLRP3, or PPARg. In some embodiments, the inflammasome associated protein is IL-1β. In some embodiments, the inflammasome associated protein in NLRP3. In some embodiments, the inflammasome associated protein is PPARg. In some embodiments, the expression or activity of IL-1β and/or NLRP3 is decreased. In some embodiments, the expression or activity of IL-1β is decreased. In some embodiments, the expression or activity of NLRP3 is decreased. In some embodiments, the expression and/or activity of PPARg is increased.

In some embodiments, the morphology of cells in a subject are altered after administration of pioglitazone, or a pharmaceutically acceptable salt thereof, and mesenchymal stromal cells to the subject. In some embodiments, the alteration in morphology comprises a reduction in inflammation. In some embodiments, the alteration in morphology comprises lower actin-bundling activity, membrane ruffling, and/or phagocytosis. In some embodiments, the alteration in morphology comprises a reduction in Ibal expression and/or activity. In some embodiments, Ibal expression and/or activity is lower after administration of PG-hMSC combination treatment than after administration of an equivalent dose of PG alone

In some embodiments, after administering pioglitazone, or a pharmaceutically acceptable salt thereof, and mesenchymal stromal cells to a subject, expression or activity of one or more pro-inflammatory cytokines and/or pro-inflammatory associated proteins is decreased, as described herein. In some embodiments, after administering pioglitazone, or a pharmaceutically acceptable salt thereof, and mesenchymal stromal cells to a subject, expression or activity of pro-inflammatory cytokines or pro-inflammatory associated proteins and anti-inflammatory cytokines or anti-inflammatory associated proteins is increased, as described herein.

In some embodiments, the one or more pro-inflammatory cytokines or associated proteins are independently selected from the group consisting of: IL-β, IL-6, IFNβ, TNFα, or CCL20. In some embodiments, the pro-inflammatory cytokines or associated protein is IL-β. In some embodiments, the pro-inflammatory cytokines or associated protein is 11-6. In some embodiments, the pro-inflammatory cytokines or associated protein is TNFα. In some embodiments, anti-inflammatory cytokines or associated proteins are PPARg or IL-10. In some embodiments, the anti-inflammatory cytokine or associated protein is PPARg. In some embodiments, the anti-inflammatory cytokine or associated protein is IL-10.

In some embodiments, one or more of IL-6, TNFα, IFNβ, and/or CCL20 gene expression decreases. In some embodiments, IL-6 gene expression decreases. In some embodiments, TNFα gene expression decreases. In some embodiments, IFNβgene expression decreases. In some embodiments, CCL20 gene expression decreases. In some embodiments, IL-6, TNFα, IFNβ, and/or CCL20 gene expressions decreases after PG, hMSC, and/or PG-hMSC combination treatments. In some embodiments, IL-6 gene expression decreases after PG, hMSC, and/or PG-hMSC combination treatments. In some embodiments, TNFα gene expression decreases after PG, hMSC, and/or PG-hMSC combination treatments. In some embodiments, IFNβ gene expression decreases after PG, hMSC, and/or PG-hMSC combination treatments. In some embodiments, CCL20 gene expression decreases after PG, hMSC, and/or PG-hMSC combination treatments. In some embodiments, IL1βgene expression decreases. In some embodiments, IL1β gene expression decreases after PG-hMSC combination treatment. In some embodiments, the gene expression decreases by about 10% to about 90% after administering pioglitazone, or a pharmaceutically acceptable salt thereof, and mesenchymal stromal cells, for example, about 10%, about 15%, about 20%, about 25%, about 30%, about 35%, about 40%, about 45%, about 50%, about 55%, about 60%, about 65%, about 70%, about 75%, about 80%, about 85%, or about 90%.

In some embodiments, PPARγ and/or IL-10 gene expressions increases. In some embodiments, PPARγ and/or IL-10 gene expressions increases after PG or PG-hMSC treatments. In some embodiments, the gene expression increases by about 10% to about 200% after administering pioglitazone, or a pharmaceutically acceptable salt thereof, and mesenchymal stromal cells, for example, about 10%, about 15%, about 20%, about 25%, about 30%, about 35%, about 40%, about 45%, about 50%, about 55%, about 60%, about 65%, about 70%, about 75%, about 80%, about 85%, about 90%, about 100%, about 105%, about 110%, about 115%, about 120%, about 125%, about 130%, about 125%, about 130%, about 145%, about 150%, about 155%, about 160%, about 165%, about 170%, about 175%, about 180%, about 195%, about 190%, or about 200%.

One skilled in the art will recognize that both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy subjects and/or those suffering from a given disorder, can be completed according to methods well known in the clinical and medical arts. Provided herein are pharmaceutical kits useful, for example, in the treatment of ARDS, which include two or more containers containing (a) pioglitazone, or a pharmaceutically acceptable salt thereof; and (b) mesenchymal stromal cells. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

A number of embodiments of the disclosure have been described. Nevertheless, it will be understood that various modifications may be made without departing from the spirit and scope of the disclosure. Accordingly, other embodiments are within the scope of the following claims.

### EXAMPLES

### Example 1: Anti-inflammatory Effects of Pioglitazone

HEK293 cells were cultured using standard methods and were stimulated with lipopolysaccharide (LPS, 500 ng/mL) in the presence of pioglitazone (20 μM) for 24 hours.

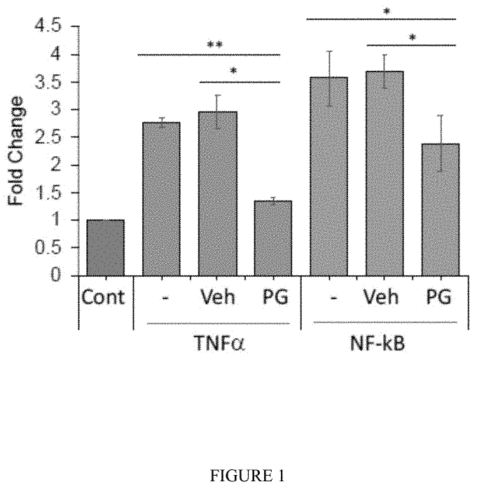

The fold change in RNA expression of TNFα and NF-kβ relative to the control culture is shown in FIG. 1. Veh: Vehicle; *p<0.05, **p<0.005.

### Example 2: PG Reduces Inflammasome Activation and Inflammatory Cytokine Production in LPS Stimulated HEK293 Cells

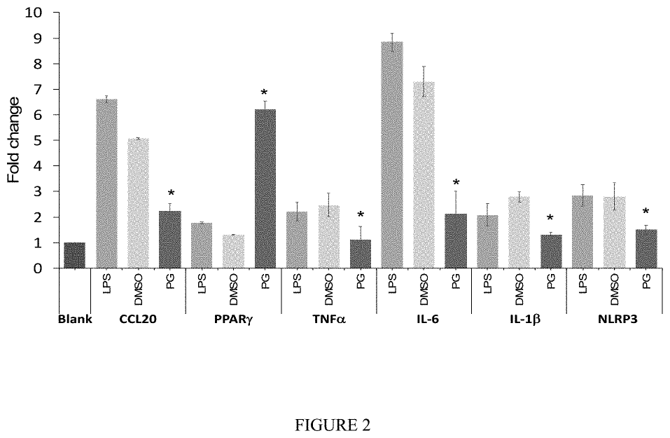

To test whether PG has anti-inflammatory activity, we stimulated HEK293 cells with bacterial lipopolysaccharide (LPS), a PAMP of gram-negative bacteria and measured expression of inflammatory cytokines (CCL20, TNFa, IL-6), and inflammasome associated proteins such as IL-lb and NLRP3. The results showed that PG treatment upregulated PPARg but reduced expression of CCL20, TNFa, IL-6, IL-lb and NLRP3 (FIG. 2).

### Example 3: PG-hMSC Combination Reduced Inflammation in LPS Stimulated IMG Cells

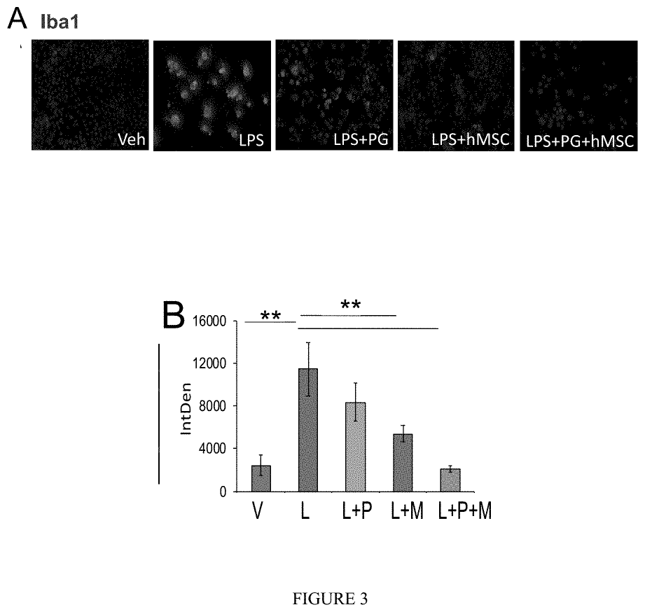

To test whether PG can act as adjunct therapy to MSC for COVID-19, we examined the effect combining PG with HMSC therapy. We stimulated mouse microglia (IMG) cells with LPS, which changed the morphology of the IMG cells and activated IMG cells. The results of staining showed increased expression of Ibal, an ionized calcium-binding adaptor protein-1 (an activation marker for microglia) (FIG. 3). PG treatment in LPS stimulated IMG cells, however, reduced intensity of Ibal expression and the number of activated cells. Moreover, human MSC (hMSC) or PG-hMSC combination treatment decreased Ibal expression significantly (FIG. 3 A, B).

### Example 4: PG-hMSC Combination Reduced Gene Expression of Inflammatory Cytokines in LPS Stimulated IMG Cells

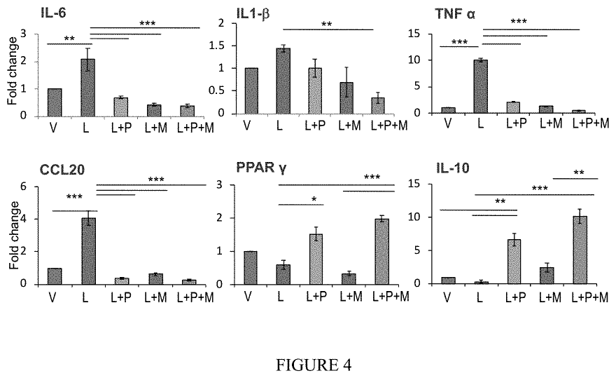

To test whether PG can act as adjunct therapy to MSC for COVID-19, we examined the effect combining PG with HMSC therapy. We stimulated mouse microglia (IMG) cells with LPS, which changed the morphology of the IMG cells and activated IMG cells. We examined fold changes mRNA expressions of a panel of pro-inflammatory (Il-b, IL-6, TNFa, CCL20) and anti-inflammatory (PPARg and IL-10) by qPCR. The results showed that IL-6, IL1-β, TNFα, CCL20 expressions increased after LPS treatment in IMG cells. However, IL-6, TNFα or CCL20 gene expressions decreased significantly 24 h after PG, hMSC or combination treatments. Of note, IL1β gene expression decreased significantly only after combination treatment. PPARγ or IL-10 gene expressions decreased significantly after LPS treatment, but PG or combination treatments increased the gene expressions significantly. Also, it was observed that, PPARγ or IL-10 gene expressions after combination treatments were significantly higher than hMSC only treatment (FIG. 4).

What is claimed is:

1. A method of treating ARDS in a subject in need thereof, comprising administering (a) pioglitazone, or a pharmaceutically acceptable salt thereof, and (b) mesenchymal stromal cells.

2. The method of claim 1, wherein the administration of (a) pioglitazone, or a pharmaceutically acceptable salt thereof, between about 3 to about 7 days prior to the administration of (b) mesenchymal stromal cells.

3. The method of claim 1, wherein between about 1 mg and 10 mg of pioglitazone, or a pharmaceutically acceptable salt thereof, per kilogram body weight thereof is administered to the subject.

4. The method of claim 1, wherein the mesenchymal stromal cells are administered intranasally.

5. The method of claim 1, wherein the mesenchymal stromal cells are administered intravenously.

6. The method of claim 1, wherein the mesenchymal stromal cells are administered intra-arterially or intra-cranially.

7. The method of claim 1, wherein the pioglitazone, or a pharmaceutically acceptable salt thereof, is administered intranasally.

8. The method of claim 1, further comprising administering (a) pioglitazone, or a pharmaceutically acceptable salt thereof, after the administration of (b) mesenchymal stromal cells.

9. The method of claim 8, wherein the pioglitazone, or a pharmaceutically acceptable salt thereof, is administered for between about 1 day to about 7 days after the mesenchymal stromal cells.

10. The method of claim 1, wherein about 0.5×10⁶ to about 1×10⁷ mesenchymal stromal cells per kilogram body weight are administered to the subject.

11. A method of treating one or more symptoms of ARDS in a subject in need thereof, comprising administering (a) pioglitazone, or a pharmaceutically acceptable salt thereof, and (b) mesenchymal stromal cells.

12. The method of claim 11, wherein the one or more symptoms are selected from: cough, shortness of breath, fast breathing, and low blood oxygen levels.

13. The method of claim 11, wherein the administration of (a) pioglitazone, or a pharmaceutically acceptable salt thereof, is between about 3 days to 7 days prior to the administration of (b) mesenchymal stromal cells.

14. The method of claim 11, wherein between about 1 mg and 10 mg of pioglitazone, or a pharmaceutically acceptable salt thereof, per kilogram body weight thereof is administered to the subject.

15. The method of claim 11, wherein the mesenchymal stromal cells are administered intranasally or intravenously.

16. The method of claim 11, wherein the mesenchymal stromal cells are administered intra-arterially or intra-cranially.

17. The method of claim 11, wherein the pioglitazone, or a pharmaceutically acceptable salt thereof, is administered intranasally.

18. The method of claim 11, further comprising administering (a) pioglitazone, or a pharmaceutically acceptable salt thereof, after the administration of (b) mesenchymal stromal cells.

19. The method of claim 18, wherein the pioglitazone, or a pharmaceutically acceptable salt thereof, is administered for between about 1 day to about 7 days after the mesenchymal stromal cells.

20. The method of claim 11, wherein about 0.5×10⁶ to about 1×10⁷ mesenchymal stromal cells per kilogram body weight are administered to the subject.
